(19) United States
(12) Patent Application Publication
(10) Pub. No.: US 2024/0148693 A1
(43) Pub. Date: May 9, 2024

(54) COMPOSITION, PREPARATION METHOD THEREFOR, AND USE THEREOF

(71) Applicant: CHANGSHA JINGYI PHARMACEUTICAL TECHNOLOGY CO., LTD, Hunan (CN)

(72) Inventor: Hailong Zhang, Hunan (CN)

(21) Appl. No.: 18/548,154

(22) PCT Filed: Nov. 29, 2021
(86) PCT No.: PCT/CN2021/133810

§ 371 Date: Aug. 28, 2023

(51) Int. Cl. A61K 31/4015; A61K 9/20; A61K 9/24; A61K 47/32

(52) U.S. Cl. CPC A61K 31/4015; A61K 9/2009; A61K 9/2013; A61K 9/2018; A61K 9/2027; A61K 9/205; A61K 9/2054; A61K 9/209; A61K 47/32

(57) ABSTRACT

A brivaracetam composition, a preparation method therefor, and a use thereof. The composition comprises brivaracetam or a pharmaceutically acceptable salt, coordination complex or hydrate thereof, and further comprises at least one selected from a swelling material, a framework material, or a sustained-release material. The composition has advantages such as a good sustained-release effect, high bioavailability, and good stability, and the sticking and picking problem present during tableting can also be avoided.

### CROSS-REFERENCE TO RELATED APPLICATION(S)

This application claims priority to Chinese Patent Application No. 202110284675.3 filed Mar. 17, 2021, the disclosure of which is incorporated herein by reference in its entirety.

### TECHNICAL FIELD

The present application relates to the field of medicine, and in particular to a composition and a preparation method therefor and use thereof.

### BACKGROUND

Epilepsy, commonly called in Chinese as [symbol] Yang Jiao Feng [symbol] or [symbol] Yang Dian Feng [symbol] , is a chronic disease of temporary brain dysfunction caused by sudden abnormal discharge of neurons in the brain. Brivaracetam (CAS No.: 357336-20-0) is a commonly used auxiliary drug for the treatment of partial-onset seizures, which is a structural analogue of antiepileptic levetiracetam with the same binding target. Brivaracetam is a selective and high-affinity ligand to the synaptic vesicle glycoprotein 2A (SV2A). It is shown by preclinical animal experiments that the affinity of brivaracetam for SV2A is 15-30 times that of levetiracetam.

The current dosage forms of brivaracetam available on the market include: oral tablets, oral solution and intravenous injection. The intravenous injection will cause pain and generally requires the assistance of professional medical care personnel, which is only performed temporarily when patients cannot be administrated orally; the oral solutions are convenient to swallow, but have poor dose accuracy; and the tablets are still the major form for use so far. At present, the brivaracetam tablets available on the market are all conventional-release tablets, and all of doses are administered twice a day; because epilepsy patients are generally accompanied by central injury, their compliance is low with multiple dosing within one day; besides, the plasma drug concentration of brivaracetam will reach the peak in a short period after administration and have large fluctuations, leading to adverse reactions or therapeutic window. Therefore, brivaracetam can be prepared into sustained-release tablets that are taken once a day to improve patient compliance and reduce fluctuations in plasma drug concentrations. However, the common sustained-release tablets only use blocking matrix materials or coating methods to slow down drug release, and have a short residence time in the upper gastrointestinal tract due to gastric emptying or intestinal transit; partial drugs are transported to the lower gastrointestinal tract before being released from the sustained-release tablets, resulting in decreased drug bioavailability and drug waste and affecting the drug efficacy.

At present, the existing gastroretentive sustained-release methods mainly include: bioadhesion, dosage size increase and flotation. Among these methods, the bioadhesion has poor selectivity, and the preparation may also adhere to the stomach contents other than the stomach wall and then be discharged from the stomach along with gastric peristalsis; the dosage size increase may be inconvenient for patients to swallow, has poor compliance, and brings the risk of pylorus blocking, affecting gastric function; the flotation generally uses light materials or gas-generating agents to make the preparation density lower than gastric juice in pursuit of the purpose, and the stability is poor. Each of these three methods has certain limitations.

In addition, brivaracetam is an extremely sticky compound (adhesion capacity). During the study, the inventor found that the brivaracetam tablet is very prone to the sticking problem during the preparation process, which brings great challenges to the appearance and content uniformity of the final product.

Therefore, it is an urgent need to find a sustained-release method that can prolong the residence time of brivaracetam preparation in the upper gastrointestinal tract and a preparation with a stable process.

### SUMMARY

### Brief Summary

In order to solve the above problems, in a first aspect, the present application provides a composition. The composition uses a swelling material, and the inventor surprisingly found that the swelling material has unexpected anti-sticking effect when used in the pharmaceutical composition of the present application, and the swelling material is unexpectedly used as an anti-sticking agent for direct compression of powders, facilitating the viscosity reduction of active ingredient and avoiding the sticking phenomenon during the tableting. In addition, by selecting suitable prescriptions and prescribed proportions in the present application, the composition can maintain the tablet shape for a long time during the dissolution, achieve gastric retention effect and good sustained-release effect, and have large swelling volume, long swelling duration, short floating-up time and long floating duration; the sustained-release effect is excellent; the bioavailability is high, and the plasma drug concentration is more smooth; in addition, it is surprisingly found in the present application that the polyvinyl acetate and polyvinylpyrrolidone used have better compatibility of the active ingredient and the excipient, and the composition is more stable in related substances. In a second aspect, the present application provides a preparation method for the composition in the first aspect. In a third aspect, the present application provides use of the composition in the first aspect in preparing a drug for treating or preventing epilepsy.

### Detailed Summary

In order to solve the above problems, the present application provides a composition, a preparation method therefor and use thereof.

In a first aspect, the present application provides a composition.

A composition comprises an active ingredient and a pharmaceutically acceptable excipient, and the active ingredient comprises brivaracetam or a pharmaceutically acceptable salt, complex or hydrate thereof, and the pharmaceutically acceptable excipient comprises at least one of a swelling material, a matrix material or a sustained-release material.

In some embodiments, the pharmaceutically acceptable excipient comprises a swelling material, a matrix material and a sustained-release material.

The matrix material may be an erosion matrix material.

The inventor surprisingly found that the sticking problem of the tableting can be effectively solved when the swelling material is used. In addition, by using the swelling material that can swell rapidly after water absorption in the composition, the tablet swells after absorbing water to a diameter exceeding the pylorus and thus cannot be discharged by the stomach smoothly; meanwhile, the tablet has a density smaller than the gastric juice after volume expansion and thus float in the stomach; the combination of swelling and flotation mechanisms is beneficial to prolong the residence time in the upper gastrointestinal tract, avoid drug waste, increase drug bioavailability, and reduce the frequency of administration. By adding the sustained-release material, the dissolution and release speed of brivaracetam, BCS class I, is slowed down, and the drug is continuously released to the absorption site, improving the bioavailability of drug. By using the erosion matrix material, when the drug action time is sufficient, the tablet will erode and gradually shrink and thus be discharged from the body, avoiding the risk of blocking the pylorus and affecting gastric function. The swelling material, the sustained-release material and the matrix material support each other in function, and work synergistically to achieve the technical effects of safety, sustained-release, long-acting and high bioavailability.

The swelling material may comprise at least one of cross-linked polyvinylpyrrolidone, sodium carboxymethyl cellulose or a cross-linked compound thereof, sodium carboxymethyl starch or a cross-linked compound thereof, and low-substituted hydroxypropyl cellulose. In some embodiments, the swelling material comprises cross-linked polyvinylpyrrolidone or cross-linked sodium carboxymethyl cellulose.

The matrix material may comprise at least one of polyvinyl acetate, polyvinylpyrrolidone, and hypromellose. In some embodiments, the matrix material is polyvinyl acetate or polyvinylpyrrolidone. In some embodiments, the matrix material is vinyl acetate and polyvinylpyrrolidone. Using polyvinyl acetate and polyvinylpyrrolidone as the matrix material is beneficial to improve the compatibility of the active ingredient and the excipient, and the sustained-release composition is more stable in related substances.

The sustained-release material may comprise at least one of polyoxyethylene, carbomer, hypromellose, and sodium alginate. In some embodiments, the sustained-release material comprises at least one of polyoxyethylene or hypromellose. In some embodiments embodiments, the sustained-release material comprises polyoxyethylene, carbomer, hypromellose or sodium alginate. In some embodiments, the sustained-release material comprises polyoxyethylene and hypromellose. In some embodiments, the sustained-release material comprises polyoxyethylene and carbomer. In some embodiments, the polyoxyethylene is a hydrophilic gel, which is prepared into a 2% aqueous solution (20° C.) with a viscosity of more than 100 mPa·s.

The active ingredient may have a content of 1 wt %-35 wt % based on the total mass of the composition. In some embodiments, the active ingredient has a content of 5 wt %-30 wt % based on the total mass of the composition. In some embodiments, the active ingredient has a content of 5 wt %-20 wt % based on the total mass of the composition. In some embodiments, the active ingredient has a content of 5 wt %-15 wt % based on the total mass of the composition. In some embodiments, the active ingredient has a content of 10 wt %-30 wt % based on the total mass of the composition.

The matrix material may have a content of 10 wt %-60 wt % based on the total mass of the composition. In some embodiments, the matrix material has a content of 10 wt %-50 wt % based on the total mass of the composition. In some embodiments, the matrix material has a content of 10 wt %-45 wt % based on the total mass of the composition. In some embodiments, the matrix material has a content of 10 wt %-40 wt % based on the total mass of the composition. In some embodiments, the matrix material has a content of 15 wt %-60 wt % based on the total mass of the composition. In some embodiments, the matrix material has a content of 15 wt %-55 wt % based on the total mass of the composition. In some embodiments, the matrix material has a content of 15 wt %-50 wt % based on the total mass of the composition. In some embodiments, the matrix material has a content of 15 wt %-40 wt % based on the total mass of the composition. In some embodiments, the matrix material has a content of 20 wt %-40 wt % based on the total mass of the composition. In some embodiments, the matrix material has a content of 25 wt %-40 wt % based on the total mass of the composition.

The swelling material may have a content of 5 wt %-60 wt % based on the total mass of the composition. In some embodiments, the swelling material has a content of 10 wt %-50 wt % based on the total mass of the composition. In some embodiments, the swelling material has a content of 10 wt %-45 wt % based on the total mass of the composition. In some embodiments, the swelling material has a content of 10 wt %-40 wt % based on the total mass of the composition. In some embodiments, the swelling material has a content of 10 wt %-35 wt % based on the total mass of the composition. In some embodiments, the swelling material has a content of 20 wt %-50 wt % based on the total mass of the composition.

The sustained-release material may have a content of 5 wt %-50 wt % based on the total mass of the composition. In some embodiments, the sustained-release material has a content of 10 wt %-45 wt % based on the total mass of the composition. In some embodiments, the sustained-release material has a content of 10 wt %-40 wt % based on the total mass of the composition. In some embodiments, the sustained-release material has a content of 10 wt %-35 wt % based on the total mass of the composition. In some embodiments, the sustained-release material has a content of 10 wt %-30 wt % based on the total mass of the composition. In some embodiments, the sustained-release material has a content of 5 wt %-40 wt % based on the total mass of the composition. In some embodiments, the sustained-release material has a content of 5 wt %-35 wt % based on the total mass of the composition. In some embodiments, the sustained-release material has a content of 5 wt %-30 wt % based on the total mass of the composition.

A dosage form of the composition may be a tablet. In some embodiments, the tablet is a sustained and controlled release tablet or a sustained-release tablet. In some embodiments, the tablet is a gastroretentive sustained and controlled release tablet or a gastroretentive sustained-release tablet.

The tablet may be a single-layer tablet comprising a sustained-release layer; or the tablet may be a bilayer tablet comprising a sustained-release layer and an immediate-release layer.

The sustained-release layer may comprise the active ingredient, the swelling material, the matrix material, the sustained-release material, an optional binder and an optional lubricant.

The immediate-release layer may comprise the active ingredient and an other excipient.

The other excipient may comprise at least one of a binder, a diluent, a disintegrant and a lubricant. In some embodiments, the other excipient comprises a diluent and a disintegrant. In some embodiments, the other excipient comprises a diluent, a disintegrant and a lubricant. In some embodiments, the other excipient comprises a binder, a diluent, a disintegrant and a lubricant.

The binder may comprise at least one of hypromellose and polyvinylpyrrolidone.

The diluent may comprise at least one of lactose or a hydrate thereof, microcrystalline cellulose, pregelatinized starch, and mannitol. In some embodiments, the diluent is lactose or a hydrate thereof.

The disintegrant may comprise at least one of cross-linked sodium carboxymethyl cellulose, sodium carboxymethyl starch, and cross-linked polyvinylpyrrolidone. In some embodiments, the disintegrant is cross-linked polyvinylpyrrolidone.

The lubricant may comprise at least one of magnesium stearate, talc, and micropowder silica gel. In some embodiments, the lubricant is magnesium stearate.

The binder in the single-layer tablet may have a content of 0-5 wt % based on the total mass of the composition.

The lubricant in the single-layer tablet may have a content of 0-3 wt % based on the total mass of the composition. In some embodiments, the lubricant in the single-layer tablet has a content of 0-2.5 wt % based on the total mass of the composition. In some embodiments, the lubricant in the single-layer tablet has a content of 0-2 wt % based on the total mass of the composition. In some embodiments, the lubricant in the single-layer tablet has a content of 0-1.5 wt % based on the total mass of the composition. In some embodiments, based on the total mass of the composition, a content of the lubricant in the single-layer tablet may be 0.5 wt %, 1.0 wt %, 1.5 wt %, 2.0 wt %, 2.5 wt % or 3.0 wt %.

The active ingredient in the sustained-release layer of the bilayer tablet may have a content of 1 wt %-35 wt % based on the total mass of the composition. In some embodiments, the active ingredient in the sustained-release layer of the bilayer tablet has a content of 5 wt %-35 wt % based on the total mass of the composition. In some embodiments, the active ingredient in the sustained-release layer of the bilayer tablet has a content of 5 wt %-30 wt % based on the total mass of the composition. In some embodiments, the active ingredient in the sustained-release layer of the bilayer tablet has a content of 10 wt %-30 wt % based on the total mass of the composition. In some embodiments, the active ingredient in the sustained-release layer of the bilayer tablet has a content of 15 wt %-30 wt % based on the total mass of the composition. In some embodiments, the active ingredient in the sustained-release layer of the bilayer tablet has a content of 20 wt %-30 wt % based on the total mass of the composition. In some embodiments, the active ingredient in the sustained-release layer of the bilayer tablet has a content of 15 wt %-25 wt % based on the total mass of the composition. In some embodiments, the active ingredient in the sustained-release layer of the bilayer tablet has a content of 24 wt %-26 wt % based on the total mass of the composition.

The binder in the sustained-release layer of the bilayer tablet may have a content of 0-10 wt % based on the total mass of the composition.

The lubricant in the sustained-release layer of the bilayer tablet may have a content of 0 -3 wt % based on the total mass of the composition. In some embodiments, the lubricant in the sustained-release layer of the bilayer tablet has a content of 0-2.5 wt % based on the total mass of the composition. In some embodiments, the lubricant in the sustained-release layer of the bilayer tablet has a content of 0-2.0 wt % based on the total mass of the composition. In some embodiments, the lubricant in the sustained-release layer of the bilayer tablet has a content of 0-1.5 wt % based on the total mass of the composition. In some embodiments, based on the total mass of the composition, a content of the lubricant in the sustained-release layer of the bilayer tablet is 0.5 wt %, 1.0 wt %, 1.5 wt %, 2.0 wt %, 2.5 wt % or 3.0 wt %.

The binder in the immediate-release layer may have a content of 0-10 wt % based on the total mass of the composition.

The disintegrant in the immediate-release layer may have a content of 1-10 wt % based on the total mass of the composition.

The lubricant in the immediate-release layer may have a content of 0-3 wt % based on the total mass of the composition. In some embodiments, the lubricant in the immediate-release layer has a content of 0-2.5 wt % based on the total mass of the composition. In some embodiments, the lubricant in the immediate-release layer has a content of 0-2.0 wt % based on the total mass of the composition. In some embodiments, the lubricant in the immediate-release layer has a content of 0-1.5 wt % based on the total mass of the composition. In some embodiments, based on the total mass of the composition, a content of the lubricant in the immediate-release layer is 0.5 wt %, 1.0 wt %, 1.5 wt %, 2.0 wt %, 2.5 wt % or 3.0 wt %.

In some embodiments of the present application, the composition comprises the swelling material, the matrix material and the sustained-release material, and based on the total mass of the composition, the active ingredient has a content of 1 wt %-35 wt %, the matrix material has a content of 10 wt %-60 wt %, the swelling material has a content of 5 wt %-60 wt %, and the sustained-release material has a content of 5 wt %-50 wt %.

In some embodiments of the present application, the composition comprises the swelling material, the matrix material and the sustained-release material, the matrix material comprises polyvinyl acetate and polyvinylpyrrolidone, and based on the total mass of the composition, the active ingredient has a content of 1 wt %-35 wt %, the matrix material has a content of 10 wt %-60 wt %, the swelling material has a content of 5 wt %-60 wt %, and the sustained-release material has a content of 5 wt %-50 wt %.

In some embodiments of the present application, the composition comprises the swelling material, the matrix material and the sustained-release material, the matrix material comprises polyvinyl acetate and polyvinylpyrrolidone, and based on the total mass of the composition, the active ingredient has a content of 1 wt %-35 wt %, the polyvinyl acetate has a content of 8 wt %-40 wt %, the polyvinylpyrrolidone has a content of 2 wt %-20 wt %, the cross-linked polyvinylpyrrolidone has a content of 5 wt %-60 wt %, and the polyoxyethylene has a content of 5 wt %-50 wt %.

In some embodiments of the present application, based on the total mass of the composition, the composition comprises 10 wt % of brivaracetam or the pharmaceutically acceptable salt, complex or hydrate thereof, 24 wt % of polyvinyl acetate, 6 wt % of polyvinylpyrrolidone, 36 wt % of crospovidone, 20 wt % of polyoxyethylene, 3 wt % of carbomer and 1% of magnesium stearate.

In some embodiments of the present application, based on the total mass of the composition, the composition comprises 10 wt % of brivaracetam or the pharmaceutically acceptable salt, complex or hydrate thereof, 32 wt % of polyvinyl acetate, 8 wt % of polyvinylpyrrolidone, 41 wt % of crospovidone, 5 wt % of polyoxyethylene, 3 wt % of carbomer and 1 wt % of magnesium stearate.

In some embodiments of the present application, based on the total mass of the composition, the composition comprises 10 wt % of brivaracetam or the pharmaceutically acceptable salt, complex or hydrate thereof, 28 wt % of polyvinyl acetate, 7 wt % of polyvinylpyrrolidone, 34 wt % of crospovidone, 15 wt % of polyoxyethylene, 5 wt % of carbomer and 1 wt % of magnesium stearate.

In some embodiments of the present application, based on the total mass of the composition, the composition comprises 10 wt % of brivaracetam or the pharmaceutically acceptable salt, complex or hydrate thereof, 28 wt % of polyvinyl acetate, 7 wt % of polyvinylpyrrolidone, 21 wt % of crospovidone, 30 wt % of polyoxyethylene, 3 wt % of carbomer and 1 wt % of magnesium stearate.

In some embodiments of the present application, based on the total mass of the composition, the composition comprises 35 wt % of brivaracetam or the pharmaceutically acceptable salt, complex or hydrate thereof, 31 wt % of polyvinyl acetate, 4 wt % of hypromellose, 20 wt % of cross-linked sodium carboxymethyl starch, 8 wt % of polyoxyethylene and 2 wt % of micropowder silica gel.

In some embodiments of the present application, based on the total mass of the composition, the composition comprises 10 wt % of brivaracetam or the pharmaceutically acceptable salt, complex or hydrate thereof, 12 wt % of polyvinyl acetate, 4 wt % of polyvinylpyrrolidone, 47 wt % of crospovidone, 25 wt % of polyoxyethylene and 2 wt % of micropowder silica gel.

In a second aspect, the present application provides a preparation method for the composition in the first aspect.

In some embodiments, a preparation method for the composition in the first aspect comprises mixing the active ingredient with the pharmaceutically acceptable excipient, and then performing wet granulation and tableting, or performing dry granulation and tableting, or directly performing tableting with powders, to obtain the composition.

In some embodiments, a preparation method for the composition in the first aspect comprises mixing the active ingredient with the matrix material, the swelling material, the sustained-release material, an optional binder and an optional lubricant, and then performing tableting to obtain the composition.

In some embodiments, a preparation method for the composition in the first aspect comprises mixing the active ingredient with the matrix material, the swelling material, the sustained-release material, an optional binder and an optional lubricant, and then performing wet granulation and pre-compression, or performing dry granulation and pre-compression, or directly performing pre-compression with powders, to obtain a sustained-release layer; then mixing the active ingredient with an other excipient and filling onto the sustained-release layer, and performing main-compression to obtain the composition, wherein the other excipient comprises at least one of a binder, a diluent, a disintegrant and a lubricant.

A preparation method for the composition in the first aspect comprises subjecting the active ingredient and an other excipient to pre-compression to obtain an immediate-release layer, and then mixing the active ingredient with the matrix material, the swelling material, the sustained-release material, an optional binder and an optional lubricant, filling onto the immediate-release layer, and performing main-compression to obtain the composition, wherein the other excipient comprises at least one of a binder, a diluent, a disintegrant and a lubricant.

In a third aspect, the present application provides use of the composition in the first aspect.

Use of the composition in the first aspect in preparing a drug for treating or preventing epilepsy.

### Beneficial Effects

Compared with the prior art, the present application has at least one of the following beneficial technical effects.

(1) When the content of matrix material is 15 wt % or more in the composition provided by the present application, the tablet can maintain the tablet shape for a long time during the dissolution, achieving the gastric retention effect and good sustained-release effect.

(2) The swelling material has unexpected anti-sticking effect when used in the pharmaceutical composition of the present application, and the swelling material is unexpectedly used as an anti-sticking agent for direct compression of powders, facilitating the viscosity reduction of active ingredient and avoiding the sticking phenomenon during the tableting.

(3) The brivaracetam gastroretentive sustained-release tablet provided by the present application can expand in volume to exceed the size of pyloric sphincter (>12 mm) within about 30 minutes or less after entering the gastric juice, and expand to 150%-300% in volume within about 1 hour, and the swelling duration is at least 4 hours.

(4) The brivaracetam gastroretentive sustained-release tablet provided by the present application has a short floating-up time and a long floating duration.

(5) The composition of the present application has a good sustained-release effect; the brivaracetam accumulative release rate of the composition of the present application is less than 30% after dissolution for 1 hour, and less than 70% after dissolution for 6 hours, and not less than 80% after dissolution for 20 hours, indicating that the composition of the present application has good sustained-release effect.

(6) The brivaracetam gastroretentive sustained-release tablet provided by the present application can achieve a 24-hour sustained-release effect in vivo, and the plasma drug concentration exposure is consistent with the control preparation; the sustained-release tablet provided by the present application has a more smooth plasma drug concentration compared with the control preparation.

(7) Compared with other matrix materials, the polyvinyl acetate and polyvinylpyrrolidone used in the present application have better compatibility of the active ingredient and the excipient, and the sustained-release tablet is more stable in related substances.

### Term Definition

In the present application, the term [symbol] room temperature [symbol] refers to the ambient temperature, which may be 20° C.-30° C.; In some embodiments, the room temperature is 22° C.-28° C.; In some embodiments, the room temperature is 24° C.-26° C.; In some embodiments, the room temperature is 25° C.

In the above description of the present application, all numbers disclosed herein are approximations, whether the word [symbol] approximately [symbol] or [symbol] about [symbol] is used or not. Based on the disclosed numbers, the numerical value of each number may have deviation by less than ±10% or a deviation reasonable to those skilled in the art, and for example, the deviation is ±1%, ±2%, ±3%, ±4% or ±5%.

The terms [symbol] be optionally selected from [symbol] , [symbol] optional [symbol] or [symbol] optionally [symbol] mean that the subsequent modified subject or condition may, not must, come into truth. For example, the [symbol] optional binder [symbol] means that a binder is either included or is not.

The term [symbol] weight percent [symbol] or [symbol] percent by weight [symbol] or [symbol] wt % [symbol] is obtained by the weight of an individual component of the composition divided by the total weight of all components of the composition and then multiplied by 100%.

The term [symbol] and/or [symbol] refers to any one of the alternatives or a combination of any two or more of the alternatives.

As used herein, the term [symbol] treatment [symbol] refers to the clinical intervention intended to alter the natural process of disease for an individual being treated. The effects include, but are not limited to, preventing disease occurrence or recurrence, relieving symptoms, reducing any direct or indirect pathological consequences, slowing disease progression, ameliorating or alleviating disease state, and relieving or improving prognosis.

In the description of this specification, the terms [symbol] one embodiment [symbol] , [symbol] some embodiments [symbol] , [symbol] example [symbol] , [symbol] specific example [symbol] or [symbol] some examples [symbol] mean that at least one of embodiments or examples in the present application includes the specific features, structures, materials or characteristics which are described with reference to the embodiment or example. In this specification, the above terms are not necessarily directed to the same embodiment or example when used illustratively. Besides, the specific features, structures, materials or characteristics can be combined in an appropriate manner in any one or more embodiments or examples. Additionally, those skilled in the art can combine the different embodiments or examples as well as the features of the different embodiments or examples in this specification, without confliction.

As used herein, the [symbol] composition [symbol] can be conveniently presented in unit dosages and can be prepared by any one of the methods well known in the pharmaceutical field. All the methods comprise the step of combining the active ingredient with the carrier which constitutes one or more accessory ingredients. Generally, the composition is prepared by uniformly and thoroughly combining the active compound with the liquid carrier, the solid particle carrier or both of them.

### BRIEF DESCRIPTION OF DRAWINGS

FIG. 1 shows drug concentration-time curves in the pharmacokinetic investigations of Example 8; in the FIGURE, curve A is the drug concentration-time curve of Example 1, and curve B is the drug concentration-time curve of a control preparation (BRIVIACT Tablets, specification: 50 mg, manufacturer: UCB).

### DETAILED DESCRIPTION

For enabling those skilled in the art to understand the technical solutions of the present application better, some non-limiting examples will be disclosed hereinafter to illustrate the present application in further details.

The reagents used in the present application are commercially available or can be prepared by the methods described in the present application.

### Detection Method for Brivaracetam and Related Substances in the Present Application

Conditions of high-performance liquid chromatography:

chromatographic column: SB-C18 column 4.6 mm×250 mm, 5 μm;

diluent: acetonitrile:water=55:45 (v/v);

mobile phase: acetonitrile:Na₂HPO₄ (0.04 mol/L, pH=2.5)=55:45 (v/v), isocratic elution;

elution time: 45 min; flow rate: 0.8 mL/min; column temperature: 30° C.; detection wavelength: 210 nm; and injection volume: 20 μL.

### Example 1: Brivaracetam Gastroretentive Sustained-Release Tablet

Prescription:

| Component | Prescribed Content (wt %) |
|---|---|
| brivaracetam | 10 |
| polyvinyl acetate | 24 |
| polyvinylpyrrolidone | 6 |
| crospovidone | 36 |
| polyoxyethylene | 20 |
| magnesium stearate | 1 |
| carbomer | 3 |

Preparation process: prescribed amounts of brivaracetam, polyvinyl acetate, polyvinylpyrrolidone, crospovidone, polyoxyethylene and carbomer were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Example 2: Brivaracetam Gastroretentive Sustained-Release Tablet

Prescription:

| Component | Prescribed Content (wt %) |
|---|---|
| brivaracetam | 10 |
| polyvinyl acetate | 32 |
| polyvinylpyrrolidone | 8 |
| crospovidone | 41 |
| carbomer | 3 |
| polyoxyethylene | 5 |
| magnesium stearate | 1 |

Preparation process: prescribed amounts of brivaracetam, polyvinyl acetate, polyvinylpyrrolidone, crospovidone, carbomer and polyoxyethylene were mixed uniformly in a mixer to obtain a preblend; the preblend was added with a prescribed amount of magnesium stearate and continued to mix in the mixer to obtain a final mixture; the final mixture was compressed into a tablet with a rotary tablet press to obtain the brivaracetam gastroretentive sustained-release tablet.

### Example 3: Brivaracetam Gastroretentive Sustained-Release Tablet

Prescription:

| Component | Prescribed Content (wt %) |
|---|---|
| brivaracetam | 10 |
| polyvinyl acetate | 28 |
| polyvinylpyrrolidone | 7 |
| crospovidone | 34 |
| carbomer | 5 |
| polyoxyethylene | 15 |
| magnesium stearate | 1 |

Preparation process: prescribed amounts of brivaracetam, polyvinyl acetate, polyvinylpyrrolidone, crospovidone, carbomer and polyoxyethylene were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Example 4: Brivaracetam Gastroretentive Sustained-Release Tablet

Prescription:

| Component | Prescribed Content (wt %) |
|---|---|
| brivaracetam | 10 |
| polyvinyl acetate | 28 |
| polyvinylpyrrolidone | 7 |
| crospovidone | 21 |
| carbomer | 3 |
| polyoxyethylene | 30 |
| magnesium stearate | 1 |

Preparation process: prescribed amounts of brivaracetam, polyvinyl acetate, polyvinylpyrrolidone, crospovidone, carbomer and polyoxyethylene were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Example 5: Brivaracetam Gastroretentive Sustained-Release Tablet

Prescription:

| Component | Prescribed Content (wt %) |
|---|---|
| brivaracetam | 35 |
| polyvinyl acetate | 20 |
| hypromellose | 4 |
| cross-linked sodium | 20 |
| carboxymethyl cellulose | |
| polyoxyethylene | 19 |
| micropowder silica gel | 2 |

Preparation process: prescribed amounts of brivaracetam, polyvinyl acetate and polyoxyethylene were mixed uniformly to obtain a preblend, the blend was granulated with a appropriate amount of hypromellose solution, and the granules were dried and screened; the granules were uniformly mixed with prescribed amounts of cross-linked sodium carboxymethyl cellulose and micropowder silica gel to obtain a final mixture; the final mixture was compressed into a tablet with a tablet press to obtain the brivaracetam gastroretentive sustained-release tablet.

### Example 6: Brivaracetam Gastroretentive Sustained-Release Tablet

Prescription:

| Component | Prescribed Content (wt%) |
|---|---|
| brivaracetam | 10 |
| polyvinyl acetate | 12 |
| polyvinylpyrrolidone | 3 |
| crospovidone | 48 |
| polyoxyethylene | 25 |
| micropowder silica gel | 2 |

Preparation process: prescribed amounts of brivaracetam, polyvinyl acetate, polyvinylpyrrolidone, crospovidone and polyoxyethylene were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Example 7: Brivaracetam Gastroretentive Sustained-Release Bilayer Tablet

Prescription:

| | |
|---|---|
| Component | Prescribed Content (wt %) |

immediate-release layer

| | |
|---|---|
| brivaracetam | 3 |
| lactose monohydrate | 15 |
| crospovidone | 1 |
| methyl cellulose | 1 |
| magnesium stearate | 1 |

sustained-release layer

| | |
|---|---|
| brivaracetam | 25 |
| polyvinyl acetate | 16 |
| polyvinylpyrrolidone | 4 |
| cross-linked sodium | 20 |
| carboxymethyl cellulose | |
| sodium alginate | 10 |
| carbomer | 3 |
| magnesium stearate | 1 |

Preparation process: prescribed amounts of brivaracetam, lactose monohydrate, crospovidone, methyl cellulose and magnesium stearate for the immediate-release layer were mixed uniformly and then pre-compressed with a tablet press; prescribed amounts of brivaracetam, polyvinyl acetate, polyvinylpyrrolidone, cross-linked sodium carboxymethyl cellulose, sodium alginate, carbomer and magnesium stearate for the sustained-release layer were mixed uniformly, and the mixture was filled onto the pre-compressed layer and main-compressed to obtain the brivaracetam gastroretentive sustained-release bilayer tablet.

### Comparative Example 1: Brivaracetam Gastroretentive Sustained-Release Tablet

Prescription:

| Component | Prescribed Content (wt %) |
|---|---|
| brivaracetam | 10 |
| polyvinyl acetate | 8 |
| polyvinylpyrrolidone | 2 |
| crospovidone | 51 |
| polyoxyethylene | 25 |
| magnesium stearate | 1 |
| carbomer | 3 |

Preparation process: the same as in Example 1.

### Comparative Example 2: Brivaracetam Gastroretentive Sustained-Release Tablet

Prescription:

| Component | Prescribed Content (wt %) |
|---|---|
| brivaracetam | 10 |
| polyvinyl acetate | 44 |
| polyvinylpyrrolidone | 11 |
| crospovidone | 10 |
| polyoxyethylene | 21 |
| magnesium stearate | 1 |
| carbomer | 3 |

Preparation process: the same as in Example 1.

### Comparative Example 3: Brivaracetam Gastroretentive Sustained-Release Tablet

Prescription:

| Component | Prescribed Content (wt %) |
|---|---|
| brivaracetam | 10 |
| polyvinyl acetate | 48 |
| polyvinylpyrrolidone | 12 |
| crospovidone | 5 |
| polyoxyethylene | 21 |
| magnesium stearate | 1 |
| carbomer | 3 |

Preparation process: the same as in Example 1.

### Comparative Example 4: Brivaracetam Gastroretentive Sustained-Release Tablet

Prescription:

| Component | Prescribed Content (wt %) |
|---|---|
| brivaracetam | 10 |
| polyvinyl acetate | 4 |
| polyvinylpyrrolidone | 1 |
| crospovidone | 60 |
| polyoxyethylene | 21 |
| magnesium stearate | 1 |
| carbomer | 3 |

Preparation process: the same as in Example 1.

### Comparative Example 5: Brivaracetam Gastroretentive Sustained-Release Tablet

Prescription:

| Component | Prescribed Content (wt %) |
|---|---|
| brivaracetam | 10 |
| polyvinyl acetate | 20 |
| polyvinylpyrrolidone | 5 |
| corn starch | 41 |
| polyoxyethylene | 20 |
| magnesium stearate | 1 |
| carbomer | 3 |

Preparation process: prescribed amounts of brivaracetam, polyvinyl acetate, polyvinylpyrrolidone, corn starch, polyoxyethylene and carbomer were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Comparative Example 6: Brivaracetam Gastroretentive Sustained-Release Tablet

Prescription:

| Component | Prescribed Content (wt %) |
|---|---|
| brivaracetam | 10 |
| polyvinyl acetate | 24 |
| polyvinylpyrrolidone | 6 |
| lactose | 36 |
| polyoxyethylene | 20 |
| magnesium stearate | 1 |
| carbomer | 3 |

Preparation process: prescribed amounts of brivaracetam, polyvinyl acetate, polyvinylpyrrolidone, lactose, polyoxyethylene and carbomer were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Comparative Example 7 to Comparative Example 11: Brivaracetam Gastroretentive Sustained-Release Tablet (Investigation of Matrix Material)

Prescription:

| | | Prescribed Content (wt %) | | | | |
|---|---|---|---|---|---|---|
| | | Comparative | Comparative | Comparative | Comparative | Comparative |
| Component | | Example 7 | Example 8 | Example 9 | Example 10 | Example 11 |
| brivaracetam | | 10 | 10 | 10 | 10 | 10 |
| matrix | polyacrylic | 30 | 0 | 0 | 0 | 0 |
| material | acid resin | | | | | |
| | hydroxyethyl | 0 | 30 | 0 | 0 | 0 |
| | cellulose | | | | | |
| | ethyl cellulose | 0 | 0 | 30 | 0 | 0 |
| | hypromellose | 0 | 0 | 0 | 30 | 0 |
| | polyvinyl | 0 | 0 | 0 | 0 | 30 |
| | alcohol | | | | | |

| | | | | | |
|---|---|---|---|---|---|
| crospovidone | 36 | 36 | 36 | 36 | 36 |
| polyoxyethylene | 20 | 20 | 20 | 20 | 20 |
| magnesium stearate | 1 | 1 | 1 | 1 | 1 |
| carbomer | 3 | 3 | 3 | 3 | 3 |

Preparation process: prescribed amounts of brivaracetam, a matrix material, crospovidone, polyoxyethylene and carbomer were mixed uniformly in a mixer to obtain a preblend; the preblend was added with a prescribed amount of magnesium stearate and continued to mix in the mixer to obtain a final mixture; the final mixture was compressed into a tablet with a tablet press to obtain the brivaracetam gastroretentive sustained-release tablet.

### Example 8: Performance Test

### 1. Investigation of Dissolution State

The brivaracetam gastroretentive sustained-release tablets prepared in Examples 1-5 and Comparative Examples 1-6 were independently added into a medium, that is, 900 ml hydrochloric acid solution with a pH of 1.2, and a rotating speed was 50 rpm, and a temperature was 37±0.5° C.; the time was recorded which the sustained-release tablet took from when it was added into the medium to tablet disintegration, so as to investigate whether the sustained-release tablet could maintain its tablet shape during the dissolution process, and the results are as follows.

| Sample | Time for maintaining tablet shape |
|---|---|
| Example 1 | >24 h |
| Example 2 | >24 h |
| Example 3 | >24 h |
| Example 4 | >24 h |
| Example 5 | >24 h |
| Comparative Example 1 | <1 h |
| Comparative Example 2 | >24 h |
| Comparative Example 3 | >24 h |
| Comparative Example 4 | <1 h |

Analysis of results: when the content of matrix material is 15 wt % or more in the tablet (see the results of Examples 1-6, Comparative Example 2 and Comparative Example 3), the tablet can maintain the tablet shape for a long time during the dissolution, achieving the gastric retention effect and good sustained-release effect; when the content of matrix material is 10 wt % or less in the tablet (see the results of Comparative Example 1 and Comparative Example 4), the tablet is prone to disintegration during the dissolution, and it is difficult to maintain the tablet shape, the gastric retention effect cannot be achieved, and the sustained-release effect is weak.

### 2. Investigation of Sticking Phenomenon

The tableting processes were observed for the sticking phenomenon in each comparative example or comparative example, and the results are as follows.

| Sample | Whether sticking phenomenon occurs |
|---|---|
| Example 1 | No |
| Example 2 | No |
| Example 3 | No |
| Example 4 | No |
| Example 5 | No |
| Comparative Example 1 | No |
| Comparative Example 2 | No |
| Comparative Example 3 | No |
| Comparative Example 4 | No |
| Comparative Example 5 | Yes |
| Comparative Example 6 | Yes |

Analysis of the results: the tableting processes of Comparative Example 5 and Comparative Example 6 have obvious sticking phenomenon because no swelling material is used; the inventor surprisingly found that the swelling material has unexpected anti-sticking effect when used in the pharmaceutical composition of the present application, and the swelling material is unexpectedly used as an anti-sticking agent for direct compression of powders, facilitating the viscosity reduction of active ingredient and avoiding the sticking phenomenon during the tableting.

### 3. Evaluation of Swelling Performance

The brivaracetam gastroretentive sustained-release tablets prepared in Examples 1-5 and Comparative Examples 2-6 were independently immersed into 0.01N HCl contained in a graduated cylinder, and the tablet volume was observed and measured regularly, and the volume expansion percentage was calculated via the following formula:

$$Volume expansion ( \% ) = \frac{Vs - Vd}{Vd} \times 100 \%$$

wherein Vd is the volume of the sustained-release tablet before being immersed in the solution (initial volume), and Vs is the volume of the sustained-release tablet after swelling at a specific time point.

Results: as shown in Table 1.

TABLE 1
Investigation results of swelling performance

| Sample | Volume expansion (%) | Swelling duration |
|---|---|---|
| Example 1 | 212 | 4.5 h |
| Example 2 | 269 | 4.5 h |
| Example 3 | 189 | 4 h |
| Example 4 | 179 | 5 h |
| Example 5 | 170 | 5 h |
| Comparative Example 4 | tablet disintegration | N/A |

The results show that the brivaracetam gastroretentive sustained-release tablets prepared in Examples 1-5 can expand in volume to exceed the size of pyloric sphincter (>12 mm) within 30 minutes or less after entering the gastric juice, and expand to 150%-300% in volume within about 1 hour, and the swelling duration is at least 4 hours.

### 4. Investigation of Floating Performance

Floating performance the brivaracetam gastroretentive sustained-release tablets prepared in Examples 1-6 were tested with reference to the second dissolution test (paddle method) of the [symbol] Chinese Pharmacopoeia [symbol] , wherein a medium was 900 ml hydrochloric acid solution with a pH of 1.2, and a rotating speed was 50 rpm, and a temperature was 37±0.5° C.; the time was recorded which the sustained-release tablet took from the beginning to floating up from the bottom of dissolution cup (recorded as the floating-up time), and the duration for the sustained-release tablet floating in the medium continuously was recorded (recorded as the floating duration), the investigation results are shown in Table 2.

TABLE 2
Investigation results of floating performance of
brivaracetam gastroretentive sustained-release tablets

| | Example | Example | Example | Example | Example | Example |
|---|---|---|---|---|---|---|
| Item | 1 | 2 | 3 | 4 | 5 | 6 |
| floating- | 3 s | 2 s | 5 s | 4 s | 3 s | 6 s |
| up time | | | | | | |
| floating | >5 h | >5 h | >5 h | >5 h | >5 h | >5 h |
| duration | | | | | | |

Analysis of the results: the brivaracetam gastroretentive sustained-release tablet provided by the present application has a short floating-up time and a long floating duration.

### 5. Investigation of In Vitro Release

The brivaracetam gastroretentive sustained-release tablets prepared in Examples 1-6 were tested independently for the accumulative release rate via the paddle method in a medium with a pH of 1.2 at 50 rpm, and the results are shown in Table 3.

A control preparation (BRIVIACT Tablets, UCB, 50 mg, batch number: 323359) was tested for the accumulative release rate via the paddle method in a medium with a pH of 1.2 at 50 rpm, and the results are shown in Table 4.

TABLE 3
In vitro accumulative release rate of brivaracetam gastroretentive sustained-release tablets

| | Example 1 | Example 2 | Example 3 | Example 4 | Example 5 | Example 6 |
|---|---|---|---|---|---|---|
| Time/h | Average/% | Average/% | Average/% | Average/% | Average/% | Average/% |
| 0 | 0.0 | 0.0 | 0.0 | 0.0 | 0.0 | 0.0 |
| 1 | 17.7 | 18.4 | 19.1 | 14.0 | 13.2 | 17.9 |
| 6 | 49.5 | 63.5 | 52.0 | 45.2 | 45.9 | 47.9 |
| 16 | 79.5 | 88.3 | 79.2 | 77.5 | 78.3 | 77.9 |
| 20 | 86.4 | 92.6 | 85.5 | 81.6 | 85.6 | 82.6 |

TABLE 4
In vitro accumulative release rate of control
preparation (BRIVIACT Tablets)

| | Control preparation |
|---|---|
| Time/min | Average/% |
| 0 | 0.0 |
| 10 | 98.8 |
| 30 | 101.0 |
| 60 | 101.4 |

Analysis of the results: the brivaracetam accumulative release rate of Examples 1-6 is less than 30% after dissolution for 1 hour, and less than 70% after dissolution for 6 hours, and not less than 80% after dissolution for 20 hours, indicating that the composition of the present application has good sustained-release effect.

### 6. Pharmacokinetic Investigation

Drug information: A: Example 1, specification: 100 mg; B: control preparation (BRIVIACT Tablets, specification: 50 mg, manufacturer: UCB)

Mode of administration: Example 1: administered once a day, administered for one day, with 12 samples; control preparation: administered twice a day, administered for one day, with 12 samples.

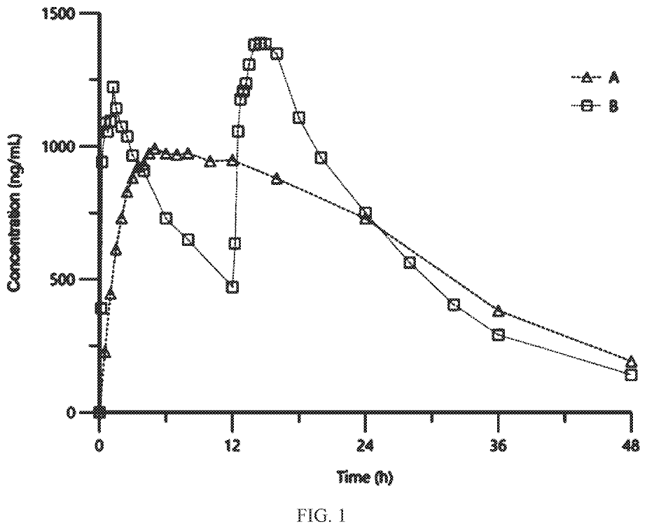

Example 1 and the control preparation are evaluated for their effectiveness via pharmacokinetic experiments. The results are shown in Table 5 and FIG. 1.

TABLE 5
In vivo pharmacokinetics of brivaracetam gastroretentive
sustained-release tablets

| | Geometric average and Ratio | | |
|---|---|---|---|
| | Preparation of | Control | |
| Pharmacokinetic | Example 1 | preparation | Ratio |
| parameters | (A, N = 12) | (B, N = 12) | (A/B)/% |
| Cₘₐₓ (ng/ml) | 1046.25 | 1653.106 | 63.29 |
| AUC_{0-∞} (ng · h/ml) | 33427.849 | 32344.723 | 103.35 |

The results show that the brivaracetam gastroretentive sustained-release tablet prepared in Example 1 can achieve a 24-hour sustained-release effect in vivo, and the plasma drug concentration exposure is consistent with the control preparation; as shown in FIG. 1, the sustained-release tablet prepared in the present application has a more smooth plasma drug concentration compared with the control preparation.

### 7. Investigation of Related Substances

The sustained-release tablets prepared in Example 1 and Comparative Examples 7-11 were kept under accelerated conditions (40° C., 75% RH) individually and detected for the related substances at 0 months, 3 months and 6 months; the results are shown in Table 6.

TABLE 6
Investigation results of related substances

| | Total impurities (%) | | |
|---|---|---|---|
| | | 3 months of | 6 months of |
| | | accelerated | accelerated |
| Sample | 0 months | conditions | conditions |
| Example 1 | 0.2 | 0.4 | 0.5 |
| Comparative | 0.2 | 0.8 | 1.5 |
| Example 7 | | | |
| Comparative | 0.1 | 0.6 | 1.2 |
| Example 8 | | | |
| Comparative | 0.2 | 0.5 | 1.0 |
| Example 9 | | | |
| Comparative | 0.2 | 0.6 | 1.3 |
| Example 10 | | | |
| Comparative | 0.2 | 0.9 | 1.7 |
| Example 11 | | | |

Analysis of the results: compared with other matrix materials, the polyvinyl acetate and polyvinylpyrrolidone used in the present application have better compatibility of the active ingredient and the excipient, and the sustained-release tablet is more stable in related substances within 6 months of accelerated conditions.

The methods of the present application have been described via the preferred examples, and it is obvious that those skilled in the art can modify or appropriately change and combine the methods and applications described herein within the content, spirit and scope of the present application to realize and implement the technology of the present application. Those skilled in the art can appropriately optimize the process parameters with the teaching from the content herein. It should be particularly pointed out that the similar substitutions and modifications are apparent to those skilled in the art and deemed to be included in the present application.

1. A composition, comprising an active ingredient and a pharmaceutically acceptable excipient, and the active ingredient comprises brivaracetam or a pharmaceutically acceptable salt, complex or hydrate thereof, and the pharmaceutically acceptable excipient comprises at least one of a swelling material, a matrix material or a sustained-release material.

2. The composition according to claim 1, wherein one or more of:
the swelling material comprises at least one of cross-linked polyvinylpyrrolidone, sodium carboxymethyl cellulose or a cross-linked compound thereof, sodium carboxymethyl starch or a cross-linked compound thereof, and low-substituted hydroxypropyl cellulose;
the matrix material comprises at least one of polyvinyl acetate, polyvinylpyrrolidone, and hypromellose; or
the sustained-release material comprises at least one of polyoxyethylene, carbomer, hypromellose, and sodium alginate; and/or the polyoxyethylene is a hydrophilic gel, which is prepared into a 2% aqueous solution (20° C.) with a viscosity of more than 100 mPa·s.

3. The composition according to claim 1, wherein one or more of:
the active ingredient has a content of 1 wt %-35 wt % based on the total mass of the composition;
the matrix material has a content of 10 wt %-60 wt % based on the total mass of the composition;
the swelling material has a content of 5 wt %-60 wt % based on the total mass of the composition; or
the sustained-release material has a content of 5 wt %-50 wt % based on the total mass of the composition.

4. The composition according to claim 1, wherein a dosage form of the composition is a tablet; and/or the tablet is a sustained and controlled release tablet or a sustained-release tablet; and/or the tablet is a gastroretentive sustained and controlled release tablet or a gastroretentive sustained-release tablet.

5. The composition according to claim 4, wherein one or more of:
the tablet is a single-layer tablet comprising a sustained-release layer; or the tablet is a bilayer tablet comprising a sustained-release layer and an immediate-release layer;
the sustained-release layer comprises the active ingredient, the swelling material, the matrix material, the sustained-release material, an optional binder and an optional lubricant;
the immediate-release layer comprises the active ingredient and an other excipient, and the other excipient comprises at least one of a binder, a diluent, a disintegrant and a lubricant;
the binder comprises at least one of hypromellose and polyvinylpyrrolidone;
the diluent comprises at least one of lactose or a hydrate thereof, microcrystalline cellulose, pregelatinized starch, and mannitol;
the disintegrant comprises at least one of cross-linked sodium carboxymethyl cellulose, sodium carboxymethyl starch, and cross-linked polyvinylpyrrolidone; or
the lubricant comprises at least one of magnesium stearate, talc, and micropowder silica gel.

6. The composition according to claim 5, wherein the binder in the single-layer tablet has a content of 0-5 wt % based on the total mass of the composition; or the lubricant in the single-layer tablet has a content of 0-3 wt %; or
based on the total mass of the composition, the active ingredient in the sustained-release layer of the bilayer tablet has a content of 1 wt %-35 wt %; the binder in the sustained-release layer of the bilayer tablet has a content of 0-10 wt %; the lubricant in the sustained-release layer of the bilayer tablet has a content of 0-3 wt %; or
based on the total mass of the composition, the active ingredient in the immediate-release layer has a content of 1-10 wt %, the binder in the immediate-release layer has a content of 0-10 wt %, and the disintegrant in the immediate-release layer has a content of 1-10 wt %; the lubricant in the immediate-release layer has a content of 0-3 wt %.

7. The composition according to claim 1, wherein the composition comprises the swelling material, the matrix material and the sustained-release material, and based on the total mass of the composition, the active ingredient has a content of 1 wt %-35 wt %, the matrix material has a content of 10 wt %-60 wt %, the swelling material has a content of 5 wt %-60 wt %, and the sustained-release material has a content of 5 wt %-50 wt %; or
the composition comprises the swelling material, the matrix material and the sustained-release material, the matrix material comprises polyvinyl acetate and polyvinylpyrrolidone, and based on the total mass of the composition, the active ingredient has a content of 1 wt %-35 wt %, the matrix material has a content of 10 wt %-60 wt %, the swelling material has a content of 5 wt %-60 wt %, and the sustained-release material has a content of 5 wt %-50 wt %; or
the composition comprises the swelling material, the matrix material and the sustained-release material, the matrix material comprises polyvinyl acetate and polyvinylpyrrolidone, and based on the total mass of the composition, the active ingredient has a content of 1 wt %-35 wt %, the polyvinyl acetate has a content of 8 wt %-40 wt %, the polyvinylpyrrolidone has a content of 2 wt %-20 wt %, the cross-linked polyvinylpyrrolidone has a content of 5 wt %-60 wt %, and the polyoxyethylene has a content of 5 wt %-50 wt %; or
based on the total mass of the composition, the composition comprises 10 wt % of brivaracetam or the pharmaceutically acceptable salt, complex or hydrate thereof, 24 wt % of polyvinyl acetate, 6 wt % of polyvinylpyrrolidone, 36 wt % of crospovidone, 20 wt % of polyoxyethylene, 3 wt % of carbomer and 1% of magnesium stearate; or
based on the total mass of the composition, the composition comprises 10 wt % of brivaracetam or the pharmaceutically acceptable salt, complex or hydrate thereof, 32 wt % of polyvinyl acetate, 8 wt % of polyvinylpyrrolidone, 41 wt % of crospovidone, 5 wt % of polyoxyethylene, 3 wt % of carbomer and 1 wt % of magnesium stearate; or
based on the total mass of the composition, the composition comprises 10 wt % of brivaracetam or the pharmaceutically acceptable salt, complex or hydrate thereof, 28 wt % of polyvinyl acetate, 7 wt % of polyvinylpyrrolidone, 34 wt % of crospovidone, 15 wt % of polyoxyethylene, 5 wt % of carbomer and 1 wt % of magnesium stearate; and/or
based on the total mass of the composition, the composition comprises 10 wt % of brivaracetam or the pharmaceutically acceptable salt, complex or hydrate thereof, 28 wt % of polyvinyl acetate, 7 wt % of polyvinylpyrrolidone, 21 wt % of crospovidone, 30 wt % of polyoxyethylene, 3 wt % of carbomer and 1 wt % of magnesium stearate; or
based on the total mass of the composition, the composition comprises 35 wt % of brivaracetam or the pharmaceutically acceptable salt, complex or hydrate thereof, 31 wt % of polyvinyl acetate, 4 wt % of hypromellose, 20 wt % of cross-linked sodium carboxymethyl starch, 8 wt % of polyoxyethylene and 2 wt % of micropowder silica gel; or
based on the total mass of the composition, the composition comprises 10 wt % of brivaracetam or the pharmaceutically acceptable salt, complex or hydrate thereof, 12 wt % of polyvinyl acetate, 4 wt % of polyvinylpyrrolidone, 47 wt % of crospovidone, 25 wt % of polyoxyethylene and 2 wt % of micropowder silica gel.

8. A preparation method for the composition according to claim 1, comprising mixing the active ingredient with the pharmaceutically acceptable excipient, and then performing wet granulation and tableting, or performing dry granulation and tableting, or directly performing tableting with powders, to obtain the composition; or
the preparation method comprises mixing the active ingredient with the matrix material, the swelling material, the sustained-release material, an optional binder and an optional lubricant, and performing tableting to obtain the composition; or
the preparation method comprises mixing the active ingredient with the matrix material, the swelling material, the sustained-release material, an optional binder and an optional lubricant, and then performing wet granulation and pre-compression, or performing dry granulation and pre-compression, or directly performing pre-compression with powders, to obtain a sustained-release layer; then mixing the active ingredient with an other excipient and filling onto the sustained-release layer, and performing main-compression to obtain the composition, wherein the other excipient comprises at least one of a binder, a diluent, a disintegrant and a lubricant; or
the preparation method comprises subjecting the active ingredient and an other excipient to pre-compression to obtain an immediate-release layer, and then mixing the active ingredient with the matrix material, the swelling material, the sustained-release material, an optional binder and an optional lubricant and filling onto immediate-release layer, and performing main-compression to obtain the composition, wherein the other excipient comprises at least one of a binder, a diluent, a disintegrant and a lubricant.

9. (canceled)

10. A method for treating or preventing epilepsy, comprising administering an effective amount of the composition according to claim 1 to subject in need thereof.
